(19) United States
(12) Patent Application Publication
(10) Pub. No.: US 2024/0009434 A1
(43) Pub. Date: Jan. 11, 2024

(54) APPLYING THERAPEUTIC AGENTS FOR GENITAL AND ANAL PRE-CANCER TREATMENT, INCLUDING CERVICAL INTRAEPITHELIAL NEOPLASIA

(71) Applicant: THE MOEIN FAMILY TRUST, San Diego, CA (US)

(72) Inventors: Sudabeh MOEIN, San Diego, CA (US); Lawrence Benjamin FISCHEL, Roseville, CA (US)

(21) Appl. No.: 18/347,478

(22) Filed: Jul. 5, 2023

Related U.S. Application Data
(60) Provisional application No. 63/367,755, filed on Jul. 6, 2022.

(51) Int. Cl. A61M 35/00; A61K 45/06

(52) U.S. Cl. CPC A61M 35/003; A61K 45/06; A61M 2205/3344

(57) ABSTRACT

Systems, methods and apparatus for genital and anal pre-cancer treatment and the treatment of cervical intraepithelial neoplasia are disclosed. One aspect includes an applicator for topically applying a therapeutic agent. The applicator includes a container to store the therapeutic agent. The applicator further includes an actuator configured to release the therapeutic agent from the container. The applicator further includes a tip configured to absorb the therapeutic agent released from the container, the tip further configured to release the therapeutic agent to a treatment area in response to contact between the tip and the treatment area. Another aspect includes an applicator for applying a film containing therapeutic material.

### CROSS REFERENCE

This application claims priority to U.S. Provisional Application No. 63/367,755, filed on Jul. 6, 2022, and is incorporated herein in its entirety by reference.

### TECHNICAL FIELD

The disclosed subject matter generally relates to genital and anal pre-cancer treatment and, more particularly, to the treatment of cervical intraepithelial neoplasia by way of the administration of chemical exfoliant agents.

### BACKGROUND

In developing countries and in socioeconomically disadvantaged areas of the world, many women lack convenient access to affordable and high-quality programs that can save their lives. Most of these women do not have access to diagnosis and treatment protocols that can cure them or prolong their lives.

According to estimates, more than one million women worldwide have cervical cancer. In 2018 alone, 569,847 new cases and approximately 311,365 deaths resulted from cervical cancer according to the World Health Organization. Nearly 90% of these deaths occurred in low-middle-income countries (LMIC). Deaths due to cervical cancer are projected to rise by almost 25% over the next 10 years without proper interventions.

Over 50% of patients diagnosed with cervical cancer die, at least in part, due to lack of availability of a safe, readily available, cost effective topical treatment of precancerous tissue. Attempts have been made to use topical acids for treatment of cervical intraepithelial neoplasia. Despite the benefits, clinicians have avoided use of acid treatment for certain practical reasons provided below.

In particular, the current application methods, which include dipping a cotton swab in a glass jar of liquid acid and applying the swab to the cervix, are impractical and dangerous. This is because the clinicians need to be confident in their application and manual dexterity to mitigate the risk of the acid dripping into the vaginal wall and eroding normal tissue.

The spillage of the liquid acid on the surrounding normal or healthy tissue, especially the posterior vaginal wall, can cause erosion through the normal tissue into the neighboring organs. As such, clinicians have been hesitant to use acid for treatment of pre-cancer due to its medical and subsequent potential legal consequences.

Furthermore, the currently available acid mixtures for treatment of cervical precancer are typically compounded in the form of liquid and applied using a cotton swab that is dipped into a vial of acid and applied onto the affected area. The clinician needs to be confident, skilled, and meticulous in performing this procedure to ensure the acid is applied to only the desired areas. Hence, this treatment method is not common among clinicians despite its efficacy due to the fear associated with the potential dripping of the acid to the undesired locations and/or surrounding normal tissue.

One major concern regarding the application of acid for treatment of cervical tissue is the increasing risk of inadvertent burn injury in the posterior cul-de-sac or vaginal wall with the potential of erosion into the patient's rectum through the vagina, due to the cervix residing at the distal end of the vaginal canal. A device known as the Wartner Pen™ is commercially available for treating non-genital warts. This and other wart treatment devices are, however, not mechanically suitable for application of exfoliating substances or acids on the cervix due to shortness in length and lack of safety features to ensure no acid is spilled on the surrounding normal tissue.

Additionally, treatment of warts or precancerous tissue on the clitoris or the clitoral hood is very challenging and requires accuracy and steady movement. Because the currently available devices are not configured to provide for the above needs, the fear of causing tissue damage or burns using strong acids, has limited the practitioner's choice of treatment to the use of topical creams like Imiquimod (Aldara or Zydara) or derivatives that are not as effective and typically cause rashes and irritation. These effects can lead to patient's dissatisfaction and non-compliance with follow up treatments leading to their increased risk of cancer.

The current method for diagnosing cervical pre-cancer or any pre-cancer in the lower genital tract or anus is colposcopy during which acetic acid is used as a staining agent to visualize abnormal/precancerous tissue. Despite its long history as a diagnostic procedure, colposcopy continues to have varying success. In conjunction with acetic acid, the sensitivity of colposcopy to distinguish normal from abnormal tissue is relatively high. The accuracy, however, to distinguish low-grade lesions from high-grade lesions and cancer remains low.

A substantial proportion of high-grade lesions may fail to be identified during colposcopy, resulting in no treatment and subsequent further growth of new cells (i.e., neoplasia). Intraepithelial growth refers to the abnormal presence of cells on the surface (epithelia) of the tissue. For example, Cervical Intraepithelial Neoplasia (CIN) may be classified according to how much epithelial tissue is affected. Low-grade neoplasia (CIN 1) refers to dysplasia that involves about one-third of the thickness of the epithelium. CIN 2 refers to abnormal changes in about one-third to two-thirds of the epithelial layer. CIN 3 (the most severe form) describes a condition that affects more than two-thirds of the epithelium.

In a post hoc analysis of more than 47,000 women, approximately 20% additional CIN 2 or worse and CIN 3 or worse were identified in women who did not have a visible lesion on colposcopy. Accordingly, the treatment of CIN 1 should be considered as important as treating CIN 2 or 3. The current treatment methods for management of patients with cervical intraepithelial neoplasia include the following treatments in order of tissue destruction and/or removal levels, starting with the least invasive and destructive method: cryosurgery, Loop Electricosurgical Excision Procedure (LEEP), laser ablation, Cold Knife Conization (CKC), and hysterectomy.

The current methods are traumatic, and are consequently not routinely prescribed for treatment of low grade lesions (CIN 1, or at times, CIN 2), largely due to the lack of a simple, non-invasive, and inexpensive procedure. Inexpensive, time-efficient, painless, and low morbidity methods and devices are needed to help improve patient care and recovery when removing tissue containing intraepithelial neoplastic cells and help substantially decrease or eliminate the risk of cervical cancer.

A gentle method of treatment is also needed for patients who are less tolerant of invasive surgical interventions. Examples of such patients are immunocompromised patients, HIV+ patients, patients on blood thinners, certain obese patients, pregnant patients with premalignant lesions, and patients of child-bearing age who have already had a surgical cervical treatment and are concerned about the impact of additional surgeries on future pregnancies.

### SUMMARY

For purposes of summarizing, certain aspects, advantages, and novel features have been described herein. It is to be understood that not all such advantages may be achieved in accordance with any one particular embodiment. Thus, the disclosed subject matter may be embodied or carried out in a manner that achieves or optimizes one advantage or group of advantages without achieving all advantages as may be taught or suggested herein.

One aspect is an applicator for topically applying a therapeutic agent to a treatment area. The applicator includes a container that stores and releases the therapeutic agent from the container. The applicator further includes a tip configured to absorb the therapeutic agent from the container and release the therapeutic agent in response to contact between the tip and the treatment area.

In some applications, the therapeutic agent includes a chemexfoliant. In some applications, the chemexfoliant includes at least one of chloroacetic acid, dichloroacetic acid, trichloroacetic acid, alpha-hydroxy acid, glycolic acid, lactic acid, tartaric acid, hydroxycaproic acid, hydroxycaprylic acid, malic acid, maleic acid, mandelic acid, beta-hydroxy acids, salicylic acid, citric acid, boric acid, boron nitride, boron carbide, other boron containing compounds, and oxalic acid. In some applications, the therapeutic agent comprises a soothing agent, such as aloe vera, allantoin, petroleum jelly, a time-release system, or lidocaine. In some applications, the therapeutic agent includes an organic agent. In some applications, the organic agent includes at least one of neem, lanolin, and a vitamin.

In some applications, the applicator includes a light source. In some applications, the light source is integrated within or attached to a body of the applicator. In some applications the light source comprises a chemiluminescent ranges. In some applications the light sources include a plurality of selectable spectral ranges. In some applications, the selectable spectral ranges may illuminate stained tissue for selective identification of tissue.

In some applications, the therapeutic agent includes at least one of a liquid, a gel, and a patch.

In some applications, the tip includes a porous, absorbent, and/or an adsorbent material. In some applications, the tip includes pores. In some applications, the tip includes at least one of a hygroscopic, hydrophilic, absorbent, and an adsorbent material, for retaining the therapeutic agent when the tip is not in contact with the treatment area. In some applications, the tip includes pores. In some applications, the tip includes at least one of a hygroscopic, hydrophilic, absorbent, and an adsorbent material, for retaining the therapeutic agent when the tip is not in contact with the treatment area. In some applications, the tip includes at least one of a dome-shaped, conical, cylindrical, chiseled, multiply curved or polygonal shape. In some applications, the tip includes a projection. In some applications, the projection includes a nipple or an extension. In some cases, the projection corresponds to a contour of the treatment area. In some applications, the tip includes a roller configured to control a release of fluid material

In some applications, the container includes one or more capsules. In some applications, the one or more capsules may include a frangible or puncturable material. In some applications, the one or more capsules may fracture or puncture in response to an applied circumferential or radial pressure. In some applications, the actuator is configured to release the therapeutic agent from each of the at least one capsules independently and separately. In some applications, the actuator is configured to release the therapeutic agent to the treatment area from each of the plurality of capsules independently and separately. In some applications, the applicator includes an auger configured to control an amount of a linear pressure. In some applications the auger includes a helical auger. In some applications, the applicator includes a puncture cap configured to puncture an end of the capsule proximate to the tip.

In some applications, the container further includes a tube for transporting therapeutic agent to or from the container to or from the tip. In some application, the actuator includes at least one of a twist end, a push end, a clicker, and a plunge end actuation mechanism.

In some applications, the container and the tip are configured for bidirectional flow of therapeutic agent between the container and the tip. In some applications, the applicator further includes a chamber configured to receive excess therapeutic agent from the tip. In some applications, the chamber includes an outer annulus, the outer annulus at least partially surrounding at least a portion of the tube. In some applications, the actuator draws by suction the excess therapeutic agent from the tip. In some applications, the actuator simultaneously releases the therapeutic agent from the container towards the tip and draws by suction the excess therapeutic agent from the tip. In some applications, the actuator includes a first actuator portion configured to release therapeutic agent from the container towards the tip, and a second actuator portion configured to draw by suction the excess therapeutic agent from the tip. In some applications, the applicator further includes a coupling that links the first actuator portion with the second actuator portion. In some applications the coupling includes toothed wheels. In some applications the coupling is configured to simultaneously cause the first actuator portion to move towards the tip and the second actuator portion to move away from the tip.

Another aspect is a method for topically applying a therapeutic agent to a treatment area. The method includes storing, in a container, a therapeutic agent. The method further includes releasing, by an actuator, the therapeutic agent from the container. The method further includes absorbing, by a tip, the therapeutic agent released from the container. The method further includes releasing, by the tip, the therapeutic agent to a treatment area in response to contact between the tip and the treatment area. In some applications, the method further includes recapture of at least some therapeutic agent from the treatment area.

Another aspect is an applicator for topically applying a therapeutic agent. The applicator includes a container configured to store a film containing the therapeutic agent. The method further includes an actuator configured to hold and release the film from the container to a treatment area in response to contact between the film and the treatment area.

In some applications, the film includes the therapeutic agent. In some applications, the film adheres to the treatment area upon contact with treatment area moisture. In some applications, the actuator includes at least one of a plunger, a twist mechanism, and a clicker configured to generate a partial vacuum to hold the film on the applicator. In some applications, the at least one of the plunger, the twist mechanism, and the clicker, is configured to release the partial vacuum to release the film from the applicator. In some applications, the actuator includes another mechanism such as a button to generate the partial vacuum to hold the film on the applicator. In some applications, the actuator includes another mechanism such as a button to release the film from the applicator.

The details of one or more variations of the subject matter described herein are set forth in the accompanying drawings and the description below. Other features and advantages of the subject matter described herein will be apparent from the description and drawings, and from the claims. The disclosed subject matter is not, however, limited to any particular embodiment disclosed.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations as provided below.

FIGS. 1 through 3, and 5 illustrate example embodiments of devices (applicators) configured for the application of therapeutic agents, such as chemexfoliation agents to treat genital neoplasia, without damaging the area surrounding the intended treatment site.

FIG. 1 illustrates a click dosing pen applicator that incorporates a click mechanism (clicker) to control the amount (dosage) of a therapeutic agent applied to a treatment area.

FIG. 2 illustrates a syringe dosing pen applicator that incorporates a push mechanism to control the amount (dosage) of a therapeutic agent applied to a treatment area.

FIG. 3 illustrates a twist dosing pen applicator that incorporates a twisting mechanism to control the amount (dosage) of a therapeutic agent applied to a treatment area.

FIGS. 4A-4C illustrate exemplary brushes for liquid or gel application.

FIG. 4A illustrates a slanted brush tip, FIG. 4B illustrates a rounded brush tip, and FIG. 4C illustrates a flat brush tip.

FIG. 4D illustrates an exemplary rounded tip that comes to a point for gel application. The tip may contain offset holes, perforations, or pores that, upon rotation, cover the desired area.

FIGS. 4E-4I illustrate exemplary felt tips for liquid application. FIG. 4E illustrates a narrow slanted tip. FIG. 4F illustrates a rounded tip with a central pointer. FIG. 4G illustrates a wide slanted tip. FIG. 4H illustrates a rounded tip that comes to a point. FIG. 4H illustrates a tip with an elongated end.

The tip shapes and configurations illustrated in FIGS. 4A-4I are non-limiting examples, as other tip shapes and configurations may include or combine dome-shaped, conical, cylindrical, chiseled, multiply curved or polygonal shapes, and holes, perforations, or pores depending on the application.

FIG. 5 illustrates a syringe dosing stamp applicator with suction that incorporates a push mechanism and coupler (toothed wheel) actuating system, to control the amount (dosage) of a therapeutic agent applied to a treatment area. The suction may recapture excess therapeutic agent from the treatment area

FIG. 6 is a cross sectional view of an exemplary implementation of a therapeutic patch applicator shaped to be received in harmony with the treatment area, such as a cervix.

FIG. 7 illustrates a ring clip-on light attachment that may be clipped on to a pen shaped applicator, such as the applicators depicted in FIGS. 1-3, 5, and 6.

FIG. 8 illustrates a c-shaped clip-on light attachment that may be clipped on to a pen shaped applicator, such as the applicators depicted in FIGS. 1-3, 5, and 6.

FIG. 9 illustrates a side attached light that may be attached to a pen shaped applicator, such as the applicators depicted in FIGS. 1-3, 5, and 6.

FIG. 10 illustrates an end ring light attachment that may be incorporated into a pen shaped applicator, such as the applicators depicted in FIGS. 1-3, 5, and 6.

FIG. 11 is a flowchart of a method for applying a therapeutic agent to a treatment area.

The figures may not be to scale in absolute or comparative terms and are intended to be exemplary. The relative placement of features and elements may have been modified for the purpose of illustrative clarity. Where practical, the same or similar reference numbers denote the same or similar or equivalent structures, features, aspects, or elements, in accordance with one or more embodiments.

### DETAILED DESCRIPTION OF EXAMPLE EMBODIMENTS

In the following, numerous specific details are set forth to provide a thorough description of various embodiments. Certain embodiments may be practiced without these specific details or with some variations in detail. In some instances, certain features are described in less detail so as not to obscure other aspects. The level of detail associated with each of the elements or features should not be construed to qualify the novelty or importance of one feature over the others.

The following embodiments are described in sufficient detail to enable those skilled in the art to make and use the invention. It is to be understood that other embodiments would be evidence based on the present disclosure, and that system, process, mechanical, or chemical changes may be made without departing from the scope of the present invention.

In accordance with some implementations of the disclosed subject matter, compositions and therapeutic films for delivering pharmaceutically active epithelial exfoliating reagents to the cervix are provided. In particular, the compositions or films can also be used to deliver trichloroacetic acid (TCA), and/or alpha-hydroxy acids (AHA) as a chemical exfoliant for treatment of cervical intraepithelial neoplasia (CIN) 1, 2, and 3. Further, this invention relates to devices for using the compositions, therapeutic films, and combinations of compositions, films, and devices which facilitate the use of these films.

Medical devices and treatment methods designed for safe application of topical treatment of physiologically acceptable liquids, gels, or dissolvable films for treatment of cervical intraepithelial neoplasia, lower genital tract, and anal pre-cancer are also disclosed. Devices may be used by other clinical professionals, such as dermatologists or estheticians, for topical application of chemical and nutraceuticals or a combination of both. The treatment can be targeted for application on any epithelial tissue anywhere in the body including the oral cavity or colon or the gastrointestinal tract. Some examples may include but are not limited to: a device containing TCA with a concentration range of 0.1% to 99% which may be used by a dermatologist as a topical treatment of cancerous or precancerous skin lesions, and a device containing TCA in a concentration range of 0.1%-99% may be used by an esthetician as a topical facial peel or skin sun damage correction. The safety and versatility of the device may further enable at home facial peels or skin wart removal.

The disclosed treatment methods and devices can also be used for treatment of vaginal warts, vaginal intraepithelial neoplasia (VAIN 1,2,3), cervical warts, cervical intraepithelial neoplasia (CIN 1,2,3), or anywhere on the external genitalia including mons pubis, clitoral hood, clitoris, labia majora, labia minora, perineum, vulvar warts, vulvar intraepithelial neoplasia (VIN 1,2,3), anal warts, anal intraepithelial neoplasia (AIN 1,2,3), penis, and scrotum. Devices designed for application of topical treatment of physiologically acceptable liquids, gels, or dissolvable films to the above-mentioned areas are disclosed.

The liquid may include a mixture of chemicals including TCA in concentrations of 0.1% to 99%, depending on intended use. The gel is composed of TCA in concentrations of 0.1% to 99% (depending on intended use) and other compounds that enhance absorption of TCA and/or potentiates its chemical effect in a synergistic fashion. The films include water-soluble or disintegrating film forming polymer and therapeutically effective amounts of chemexfoliation agents. Based on scientific experience, concentrations of TCA for the cervix, vaginal wall, and external genitalia may, for example, be 90%, 80-85%, and 50%, respectively. Other concentrations TCA in the 0.1% to 99% range may be employed depending on the desired therapeutic outcome.

The disclosed devices are also designed to facilitate safe application of chemexfoliation agents including chemical compounds such as TCA, AHA, Retin-A, Podophyllin resin, Podofilox lotion or gel. Devices are designed to facilitate safe application of organic (herbal) compounds such as Neem, Lanolin, and High Dose Vitamin C of up to 1.5 g/kg. Various combinations of devices and films, liquids, or gels, can be made, based on intended use, and can sufficiently comprise a kit to enable treatment of precancerous lesions in the above-mentioned areas in the human body.

In accordance with certain embodiments, applicators are provided for the safe treatment of cervical intraepithelial neoplasia, lower genital tract lesions, anal warts, anal intraepithelial neoplasia, for the administration of key chemexfoliation agents, and more particularly, compositions of liquids, gels, or therapeutic films, or any other chemical composition, for delivering pharmaceutically active or organic epithelial exfoliating reagents to the areas mentioned in abstract.

The combination of the chemical or natural agents can also be used to deliver AHAs or TCA, Neem, Retin-A, as a chemical or natural exfoliate for treatment of CIN 1, 2, 3, or VIN 1, 2, 3, or VAIN 1, 2, 3, or AIN 1, 2, 3. Also disclosed are devices for application of liquid, films, and gels which facilitate a safe delivery and application of the compounds with minimal or without any risk of spilling or leakage of the material outside the area designated for treatment.

The example embodiments, different activation and deactivation methods and mechanisms are provided, including but not limited to actuators that function by way of clicking a mechanical button (clicker), twisting a knob, or push mechanisms. In one aspect, the actuator functions by way of the manual application of circumferential or radial pressure around a flexible silicone or plastic body which breaks an interior thin-glass (or chemically resilient material to acid) container filled with chemexfoliation agents. The applicators can have bidirectional flow capabilities of chemexfoliation liquid minimizing the risk of chemexfoliation liquid spillage onto undesired areas. The applicator may have a non-slip exterior surface and may be ergonomically designed for added ease of use for the clinician performing the treatment.

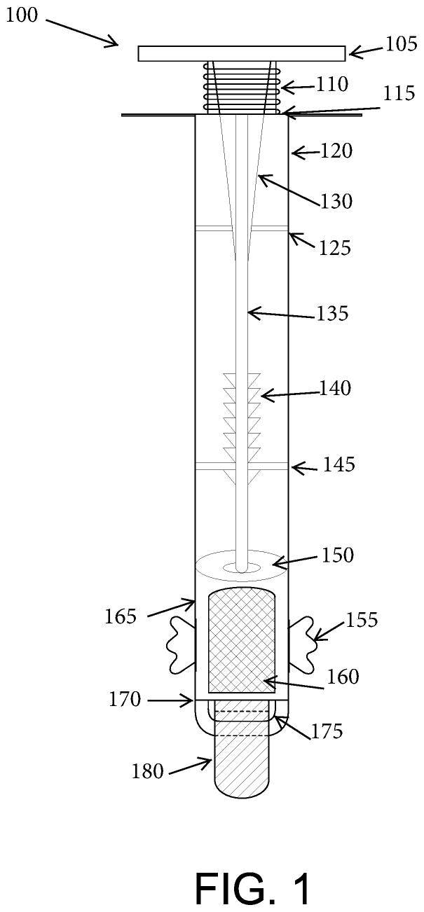

Referring to FIGS. 1 through 10, in accordance with one or more embodiments, systems, methods, and devices are provided for the application of chemexfoliation agents or acid-based compositions, such as TCA, for genital neoplasia and for bypassing the risk of damaging the area surrounding the intended treatment site. Provided are applicators or devices for delivering compositions for treatment of cervical intraepithelial neoplasia or any other pre-cancer of lower genital tract or anus. The applicators or devices may be used for a single procedure, and disposed of after use. Exemplary applicators or devices may include one or more of a felt tip, a brush tip, an applicator, a painter, a dispenser, a rotating disperser, a stamper, or a ballpoint roller.

The ectocervix is convex and the squamocolumnar junction is concave and ends in the cervical os which leads to the cervical canal. TCA may be used, in accordance with one embodiment, in the denaturation or precipitation of macromolecules, such as proteins, DNA, and RNA. TCA may be used in cosmetic treatments such as chemical peels, as well as topical medication for chemo-ablation of warts, including genital warts. It is considered safe for use for this purpose during pregnancy or in HIV positive patients.

In accordance with some embodiments, an applicator or device, such as that disclosed in FIGS. 1 through 10 is configured to deliver a predetermined dose or fraction thereof of chemexfoliating reagent to the epithelial dermis of the cervix. The chemexfoliating reagent is selected from chloroacetic acid, dichloroacetic acid, trichloroacetic acid (TCA), alpha-hydroxy acid (AHA), glycolic acid, lactic acid, tartaric acid, hydroxycaproic acid, hydroxycaprylic acid, malic acid, maleic acid, mandelic acid, beta-hydroxy acids, salicylic acid, citric acid, boric acid, boron nitride, boron carbide, other boron containing compounds, and oxalic acid. Effective concentrations of TCA or other acids in the treatment layer may be equivalent to between 0.1% and 99% w/v solutions, such as between 60% and 90% w/v, for example.

In one embodiment, if AHA is used in the treatment solution, the amount of AHA dispensed by the applicator is the equivalent of a volumetric dose of approximately 0.1 milliliter to 5 milliliters in 0.1 milliliter increments of 0.1% to 99% AHA w/v solution. The substrate is chemically compatible with TCA (i.e., non-reactive with the TCA), or whichever chemexfoliating agent is chosen to be dissolved in the substrate material. Organic (herbal) agents may include Neem, Lanolin, High Dose Vitamin C of up to 1.5 g/kg, for example.

Soothing agents may include aloe vera and allantoin and petroleum jelly and lidocaine. Allantoin has a moisturizing and keratolytic effect, increasing the water content of the extracellular matrix and enhancing the desquamation of upper layers of dead skin cells, increasing the smoothness of the skin; promoting cell proliferation and wound healing; and a soothing, anti-irritant, and skin protectant effect by forming complexes with irritant and sensitizing agents.

Depending on implementation, allantoin may expedite recovery of the new skin cells, and shorten the time between TCA treatments. Allantoin ameliorates the wound healing process, by modulating the inflammatory response, and promotes fibroblast proliferation and synthesis of the extracellular matrix. This will serve to shorten the time interval between treatments, and to promote recovery that is more complete. Concentrations of allantoin may range from 1% w/v to 10% w/v, for example. Other agents may include antiviral medications like lopinavir.

In certain embodiments, a pen delivery mechanism such as that shown in FIGS. 1 through 3 may be utilized depending on the intended use. The device length for application to the cervix and/or vagina (intravaginal application) may, for example, be in the range of 3 to 12 inches in length based on the length of the vaginal canal which leads to the cervix (application site). If the pen is used for external genitalia, anus, and anal canal the length of the pen can be anywhere from 1 inches to 12 inches, for example.

An example applicator (pen device) may have three different parts: the tip, the container which holds the chemexfoliation fluid, and the activator which initiates the release of the chemexfoliation fluid into the tip of the applicator for immediate application on pre-cancerous tissue. The applicator itself, prior to containing the chemexfoliation fluid, may be configured in the shape of a hollow cylinder made, for example, of metal, plastic, wood, silicone, or other various compounds that are safe and/or sterile.

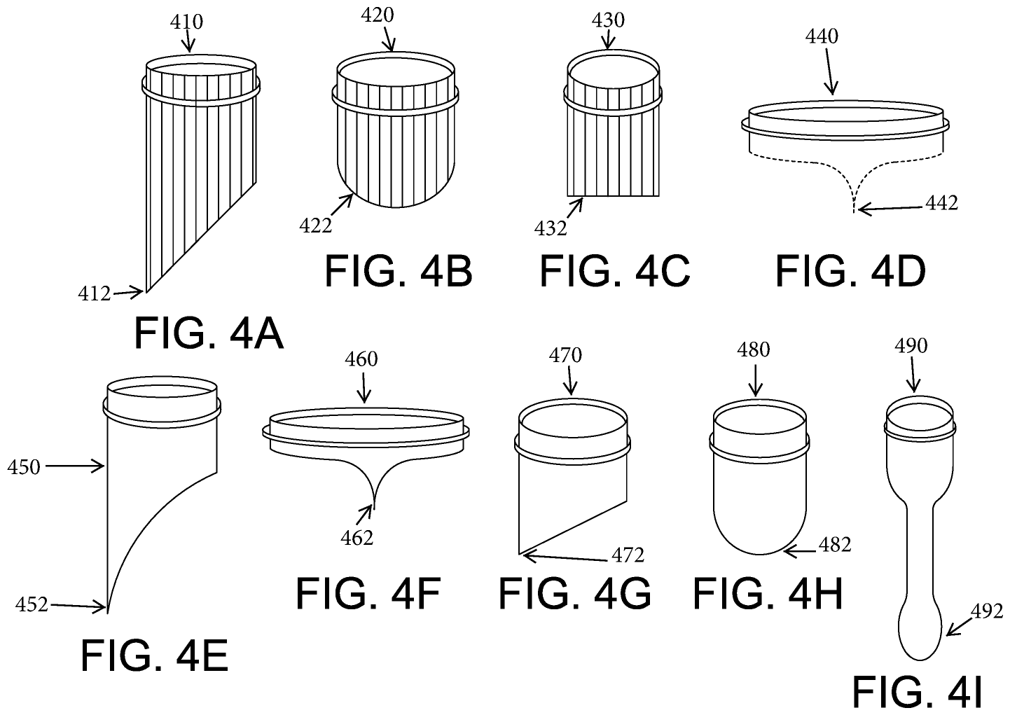

Applicator tip components may be detachable from the applicator container holding the chemexfoliation agent(s). The removable applicator tip can be replaced with other more suitable applicator tips, as shown in FIG. 4 for example, to complement the intended method of use. Various embodiments may have an integrated light source with different color bulbs or a bulb that can project different colors using a color filter, for example. The light source can be battery operated or chemiluminescent. The light can be of various colors and have various wavelengths in the spectral ranges of 300 nm to 700 nm, 495 nm to 625 nm, and 300 nm to 495 nm, for example. This will allow visualization of the abnormally stained tissue at the time of treatment. An example light source can be especially useful when the applicator is used in settings where proper light is scarce or lacking. An example light source can be especially useful when the applicator is used in conjunction with staining agents (acetic acid, Lugol's solution, fluorescein sodium) or coloring agents for identification of abnormal tissue and differentiation from normal tissue. The applicator tip according to the present invention may be utilized in manual, robotic, or automated applications.

As shown, applicator tips may have a variety of suitable shapes, including but not limited to dome-shaped, conical, cylindrical, chisel, multiply curved, or polygonal shapes. All listed shapes may include a nipple-like shape or extension at the middle most area, or other position, of the applicator tip. For example, the tip may be a tube, or comprise a rolling ball, stamper, brush, swab, or similar tip. In one example, a dome-shaped applicator tip with nipple may be used for the application of TCA treatment on the cervix, where the tip of the nipple, as shown in FIG. 4, has a projecting point, either sharp or rounded at the distal end, to match the contour of the treatment area and allow the clinician or operator to fully engage the treatment area using a single application with or without having to rotate or move the applicator in multiple directions.

Referring to FIG. 4D, for example, the syringe applicator may disperse the gel through holes staggered over the surface of the applicator head. The applicator head fits on the cervix with the central extension entering the central opening of the cervix. Coincident rotation of the applicator and dispensing of the gel (e.g., by way of pressing down on the plunger) through the staggered holes ensures that the entire surface of the cervix is lathered with the gel. The staggering of the holes is such that upon rotation, most if not every part of the cervix is covered. The volume of the syringe is calibrated to ensure that the targeted amount of material is delivered. The syringe and applicator head could be a single piece (i.e., uniconstructional) or separate pieces (multistructural) that fit together.

The applicator tip may be composed of any of a variety of materials including polymerized materials such as plastics, foams, rubber, thermosets, films or membranes. Additionally, the applicator tip may be composed of materials such as metals, glass, paper, ceramics, cardboard, polycaprolactone and the like. The applicator tip may be porous, absorbent, or adsorbent in nature to enhance and facilitate loading of the material on or within the applicator tip. For example, the applicator tip may be composed of a material having random pores, a honey-comb material, a material having a woven pattern, etc. The degree of porosity or the size of the openings will depend on the materials being used. The material used will depend on the intended treatment application site. These variations make it possible to use different compounds: chemicals and nutraceuticals, or a combination of both. Depending on the viscosity of the compounded material that gets put in the pen for respective purposes the shape of the tip and the consistency of the material used will need to be designed in order to absorb and transfer the compound from the pen or applicator to the tissue.

The applicator container, according to certain embodiments, may be configured in a variety of shapes and sizes depending on intended use. The applicator container may have an elongated tubular portion. For example, for application of limited amounts of TCA, the applicator container may be a syringe, a tube, a vial, a bulb, or a pipette. In one example, a closed tube having frangible properties may be provisioned with TCA and inserted in a flexible container as shown in FIGS. 1-3. This would provide a type of applicator container for the application of TCA treatment on the cervix, which may be manipulated by way of pressure exerted by a user to the frangible tube. Exertion of pressure through the flexible container to the frangible tube may cause the TCA contained in the tube to flow out into the container and exit through a tip attached to the flexible container. The applicator container may have features (tabs, hooks, nooks, projections, or other attachment features) enabling the attachment of light sources, lenses, or mirrors in parallel with the applicator. These attachments may facilitate the illumination, location, or application of the target sites.

In certain aspects, an applicator may be configured to control the flow of the TCA in and out of the container in a bidirectional manner, for example, through a pressure gradient, vacuum or a battery-operated release and suction system that allows proper expulsion of the chemexfoliation chemical (organic or non-organic). This design is to facilitate and control the flow of the desired chemicals to the tip of the applicator while simultaneously withdrawing the excess chemexfoliation chemicals. This bidirectional flow capability will prevent excess fluid from coming in contact with the normal surrounding tissue and prevents injury to sensitive areas in lower genital tract, including the cervico-vaginal junction, vaginal posterior cul-de-sac, or posterior vaginal epithelium, or posterior aspect of the anal opening. This added feature will address limitations of current products that provide limited precision in localized applications and also provides the treating clinician or device operator with additional dexterity and comfort in applying the treatment with precision and accuracy.

Applicator embodiments can have different actuators or activation methods to expel chemexfoliation agents from the applicator container into the applicator tip. These include but are not limited to twist end, push end, and plunge end mechanisms or actuators. Referring to FIGS. 1-3, for example, the actuation mechanism may include a silicone tube that houses a capsule (ampule) made of frangible glass, and upon manual circumferential pressure around the silicone tube the frangible glass breaks and releases the chemexfoliation agent out to the applicator tip for immediate intended use. The silicone tube and housed frangible glass unit can be located at the distal, mid, or proximal end of the applicator depending on intended use location. For example, the silicone tube and housed frangible tube location for chemexfoliation chemical application on the cervix may be in the mid two-thirds or lower one-third of the ergonomically built, anti-slip applicator.

The applicator may, for example, be part of a TCA treatment kit. An example kit tray may accommodate the equipment and reagents for performing a therapeutic treatment. This kit may, for example, contain at least one of a disposable speculum, medium sized gloves, lubricating gel sachet, under buttocks drape, a staining agent (e.g., Acetic Acid, Lugol's iodine solution, Fluorescein Sodium solution) in a sachet, dedicated container, or pipette with screw cap (e.g., 5 cc, 4% Lugol Iodine solution), TCA applicator, large tip cotton swabs, and a feminine pad. A written instruction or patient handout on what to expect after a TCA treatment may be also included. The components may be in a bag, container, or fixed position mold to form the kit. The kit may be vacuum formed and includes a sealed peelable cover for sealing the kit tray during storage and transport. The items may be conveniently provided and arranged to facilitate a comprehensive therapeutic treatment session during the office visit.

A kit may be used for cervical intraepithelial neoplasia to treat a target area every two to seven days for a total of four to six visits. The method by which the kit may be used may include placing the patient in a dorsal lithotomy position. The speculum from the kit is inserted into the patient's vagina to expose the patient's cervix. A sterile swab may be used to remove vaginal discharge or secretions on the cervix, visualizing the cervix for gross abnormalities. Lugol's iodine solution or other staining solutions may be applied to the patient's cervix using the prefilled pipette, or combination of pipette and solution vial, in the kit.

Excess amount of Lugol's solution (or other staining agents) in the vaginal posterior cul-de-sac are removed by using the pipette. Lugol's solution or other staining agents may be applied multiple times in order to stain the patient's cervix in all areas to delineate the normal tissue from the abnormal tissue on the cervix. Once adequate staining is obtained, the excess Lugol's or other staining solutions in the posterior cul-de-sac is collected with an empty pipette and discarded. The TCA applicator will then be used for treatment of the target area on the cervix. Any excess liquid or discharge will be removed by using cotton tip swabs, syringe, or alternate methods.

Once the clinician is satisfied with the treatment, the speculum is removed from the patient's vagina. Patient's legs are gently moved out of the stirrups onto the bed. Patient is observed for about 5 minutes while laying on the bed. If the patient is comfortable and has no dizziness or extreme pain, she is assisted to sit up on the bed with her leg hanging from the bed first and gently stepping off the bed with assistance. A clean wrapped feminine pad from the kit is given to the patient for use. A patient education pamphlet instructing precautions and describing what to expect after TCA treatment is handed to the patient prior to discharge. Kits may include the same or all of the above components, or equivalents, or additional components.

Referring to FIGS. 1 through 4, an example applicator and different applicator parts are illustrated. In this example embodiment, the clinician activates the flow of liquid chemexfoliation agents by twisting the top portion of the pen, screwing down into the capsule, and pushing the capsule down into the top of the sharp cylinder. The cylinder pierces a hole at the bottom of the capsule allowing liquid TCA to flow in a controlled manner into the tip of the pen. The pen-shaped applicator is accompanied by detachable tips that twist into the body of the pen. The clinician may choose the tip based on how and where they intend on distributing the liquid TCA.

FIGS. 1-4 illustrate example applicators and different applicator parts. The clinician activates the flow of liquid chemexfoliation agents by pressing the sides of the pen to crack the frangible capsule. Then, by “clicking” with a clicker, twisting, or pressing the top portion of the pen exerts pressure on the capsule, and pushing exfoliant agent, for example, liquid TCA, in a controlled manner, towards the tip of the pen. The pen-shaped applicator is accompanied by detachable tips (FIG. 4) that may twist into the body of the pen. The clinician may choose the tip based on how and where they intend to apply the liquid TCA.

FIG. 1, illustrates a click dosing pen applicator, 100. The operator expels material using the clicker. Depressing on the plunger causes grippers, the flexible push rods 130 within the solid container wall 120 in FIG. 1, to advance the plunger rod 135 such that one or more anchors 140 project beyond the brace 145 to expel a controlled amount of fluid. The brace 145 prevents the plunger rod 135 from retracting. The spring coil 110 repositions the push top 105 to enable the next click action. When not pressed, the flexible push rods 130 do not grip the plunger rod 135.

When the operator is ready to expel the fluid, the release button 155 on the flexible wall container 165 is pressed to break the fluid-containing ampule 160. The operator uses the plunger rod 135 to push down the plunger tip/head 150 on the ampule 160. The brace/retainer 170 controls the amount or rate of therapeutic fluid expelled. The fluid is expelled out of the applicator 100 onto the desired location of the patient using a felt tip 180 which is attached on the applicator using a tip attachment base 175 attached to the brace 170.

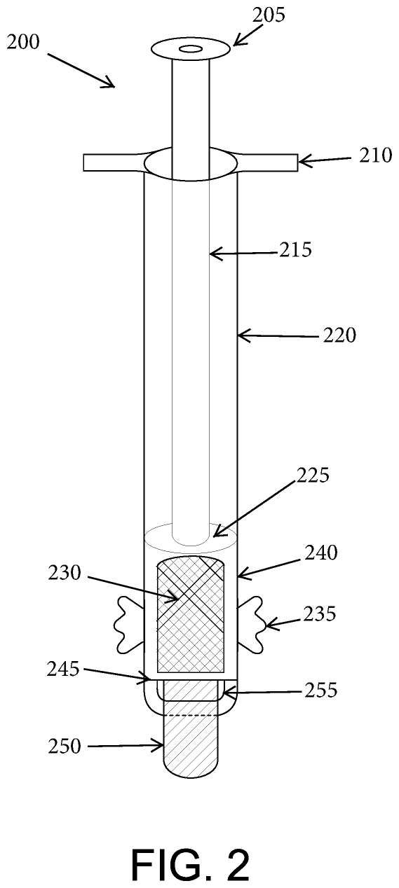

FIG. 2 illustrates a syringe dosing pen applicator 200. The operator expels material using the syringe. Depressing on the push top 205 by gripping onto handle 210 causes the piston 215 within the container wall 220, to advance the plunger head 225. When the operator is ready to expel the fluid, the release button 235 on the flexible wall container 240 is pressed to break the fluid-containing ampule 230. The release buttons 235 can flex or move inward in response to operator pressure on the release button 235 putting strain on the fluid-containing ampule 230. In response to the strain applied, the fluid-containing ampule 230 will crack or break, releasing the therapeutic fluid therein. The fluid-containing ampule 230 can be similar to those described elsewhere herein.

The operator then uses the piston 215 to push down the plunger head 225 on the ampule 230. The brace/retainer 245 controls the amount of fluid expelled. The fluid is expelled out of the applicator onto the desired location using a felt tip 250 which is attached on the applicator using a tip attachment base 255 attached to the brace 245.

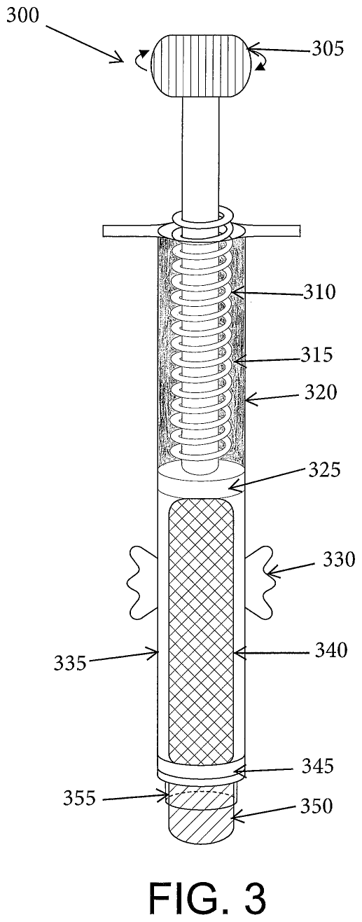

FIG. 3, illustrates a twist dosing pen applicator, 300. In FIG. 3, the top portion of the applicator is connected to a mechanism that upon manual twisting in a clockwise motion activates the controlled release of the liquid to the tip of the applicator for immediate application. The operator expels material by twisting top 305 which rotates to advance the screw 310 in clockwise direction. Twisting the twist top 305 causes screw to pass through a support member 315 contained within or connected to the solid container wall 320. When the operator is ready to expel the fluid, the release button 330 on the flexible wall container 335 is pressed to break the fluid-containing ampule 340. The operator then twists the twist top attached to a plunger rod 325 to push down on the ampule 340. The brace/retainer 345 controls the amount of fluid expelled. The fluid is expelled out of the applicator 300 onto the desired location using a felt tip 350 which is attached on the applicator using a tip attachment base 335 attached to the brace 345. The structure and function of applicator 300 and its operation can be similar to those described elsewhere herein.

FIG. 4, illustrates different types of liquid and gel tips of the pen applicator. FIGS. 4A-4C describes different shaped brush applicator tips for fluid application, including liquid, cream, gel, and other similar fluids. The different embodiments of applicator tips can be variously used for application of the chemexfoliation fluid to various desired locations.

In FIG. 4A, the brush 410 is asymmetric in shape with a precision tip 412 for very precise application of the fluid. In FIG. 4B, the brush 420 has a round applicator head or bristles 422 for the gel application of the fluid. In FIG. 4C, the brush 430 has a flat applicator head or bristles 432 for the gel application of the fluid. FIG. 4D describes a porous, concave cup-shaped applicator tip 440 for gel application made from pierced plastic to conform to the generally convex or toroidal shape of the cervix during the application of the fluid. The applicator tip 440 has a sharp end 442. FIGS. 4E-4I describes different shaped felt applicator tips for liquid application of the chemexfoliation fluid to the desired locations. The felt tip may be made of highly compressed synthetic fibers or porous ceramics. In FIG. 4E, the felt 450 is concave in shape with a precision tip 452 for precise liquid application of the fluid. In FIG. 4F, the felt 460 is concave cup-shaped a sharp end 462 to conform to the generally convex or toroidal shape of the cervix during the liquid application of the fluid. In FIG. 4G, 470 is an angled, straight-edge felt applicator with a precision tip 472 for precise liquid application of the fluid. In FIG. 4H, the felt 480 has a round applicator head 482 for the liquid application of the fluid. In FIG. 4I, the felt 490 is cylindrical in shape which tappers into a bulb-shaped applicator head 492 for the liquid application of the fluid.

Brush applicators can be advantageously used for cream or gel applications, or higher viscosity fluids, and head applicators can be used for liquid applications or lower viscosity fluids. A person of skill in the art would understand how to use different shaped applicator tips for precise and accurate application of a therapeutic fluid to specific body parts or body locations.

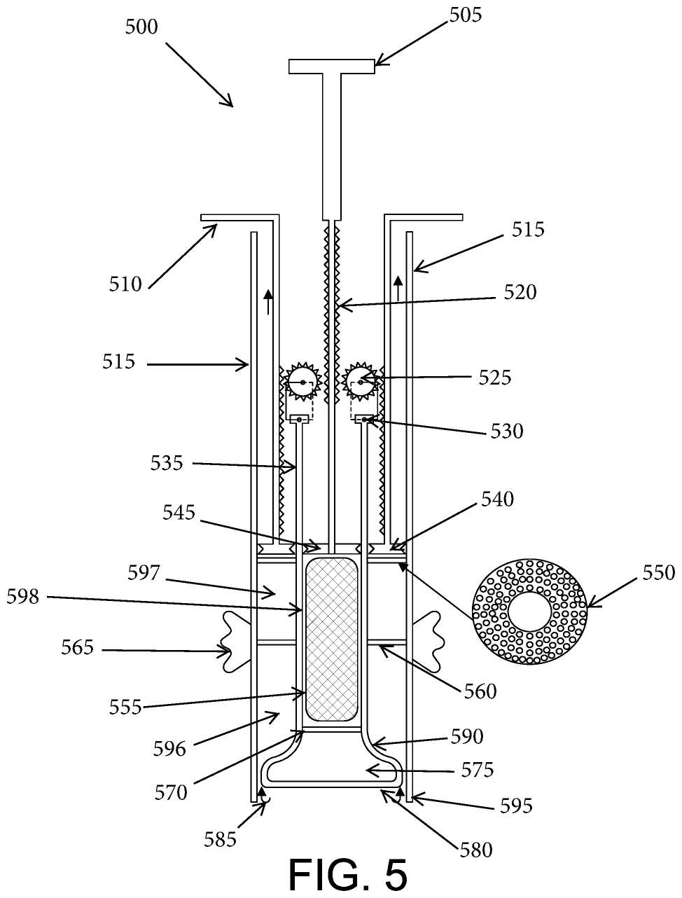

FIG. 5, illustrates an exemplary applicator 500 with two concentric tubes forming an inner core 535 and an outer annulus, 515 the shell. The inner core 535 contains the therapeutic agent by whichever production method is chosen. The inner core 535 is compressed by a plunger actuated (pressed) thumb presser 505 by the user's thumb. The outer annulus 515 provides a chamber into which to draw (by suction) the excess therapeutic agent after the therapeutic agent contacts the target area for treatment. The inner and outer plungers are connected by a toothed wheel (gear) 525, or other coupling, which forces the plungers to move together. The outer annulus chamber is filled with the excess therapeutic agent as the plunger is actuated by the user's index and middle fingers. The disk meshes, 550 and 560, are fixed in position by attachment to the inner core, 555, and the outer annulus 515, either with brackets or by weld or by being of the same molded part.

The outer annulus handles 510 could be either rings into which the index and middle fingers enter or tabs onto which the index and middle fingers can pull, similar to a syringe. The teeth on the plungers 520 are designed to ensure that they move together (inner plunger toward tip, outer plunger away from tip) and the gearing is configured to specify the relative distance moved. The cavity or suction space created in the outer annulus 515 can advantageously be as much as the compressed space in the inner cylinder to ensure capture and retention of the therapeutic agent after contact with the target area. The toothed wheel 525 is anchored to the shell 530, which does not move, that is, with respect to which the two plungers move in opposite directions. The inner plunger is anchored to an inner rubber septum 545 placed on the proximal end of the inner core 555. The outer plungers are anchored to an outer rubber septum 540 placed on the proximal end of the upper side chamber 597. A first disk mesh 550 is placed between the outer rubber septum 540 and the upper side chamber 597. The distal end of the upper side chamber is connected to the lower side chamber 596 using a second disk mesh 560 to crack the inner core 555.

When the operator is ready to expel the therapeutic agent, the release button 565 on the outer annulus 515 is pressed and the disk mesh 560 pushes against the container wall around the ampoule 598 to break the ampoule 598. The operator uses the thumb presser 505 to push down the inner plunger on the ampoule 598. The brace/retainer 570 controls the amount of therapeutic agent expelled. The brace/retainer 570 includes a curved portion extending into the lower side chamber 596. The curved portion holds a porous mesh 590. Upon operation of the plunger, The fluid is expelled onto a porous mesh 575 placed between the brace, the curved pieced to hold the porous mesh 590, and the full mesh bridge 580. The full mesh bridge 580 is similar to 550 but is a full disk, not having an annulus. The outermost walls 595 of the outer annulus 515, labeled 595, will abut the cervix circumferentially to ensure that no liquid travels beyond the region covered by the applicator, thus preventing dripping of the liquid. Excess fluid can be drawn into a space between the curved portion of the brace/retainer 570 and the outer annulus 595 at locations indicated by small curved arrows 585.

The excess fluid is drawn through the spaces due to the action of the outer plungers moving upward in response to the inner fluid moving downward.

The internal periphery 585 of the bottom 595 of the outer annulus 515 that abuts the cervix can advantageously contain absorbent material (dry cotton or sponge, possibly with acid neutralizing agents) to absorb any excess liquid.

Referring to FIGS. 1 through 3 and 5, the midsection or body of the applicator holds the thin-glass capsule that contains the TCA. A sharp structure within a twist cap portion of the tip can puncture the capsule in response to an operator applying a force, thus activating the dispensing of liquid TCA into the tip of the pen for application on the treatment site. Alternatively, the capsule may be made of a frangible material that fractures and crumbles to release the TCA liquid. This acts as the control flow or controlled release mechanism.

Referring to FIGS. 1 through 3 and 5, the capsule may be made of material that blocks light and is resistant to corrosion from the acid within the capsule. Possible materials include glass, plastic, waxy solids, or other materials, including coated materials.

Referring to FIGS. 4 and 5, the various applicator tips can be felt, brush, a rolling ball, or film

In an example embodiment, the film can be made from Polycaprolactone (PCL) which is a biodegradable polyester with a low melting point of around 60° C. and a glass transition temperature of about −60° C. Polycaprolactones impart good resistance to water, oil, solvent and chlorine to the polyurethane produced and is a polymer that may be used as an additive for resins to improve processing characteristics and end use properties (e.g., impact resistance). Being compatible with a range of other materials, PCL can be mixed with starch to lower its cost and increase biodegradability or it can be added as a polymeric plasticizer to polyvinyl chloride (PVC), in accordance with one or more embodiments.

The treatment layer surface equipped with the film may be hydrophilic and/or hygroscopic to enable self-attachment of the film at the time of application such that the film starts dissolving upon contacting any moist epithelial surface, including the vaginal walls. It may be desirable to isolate the treatment layer's exposed surface from intermediate epithelial surfaces upon insertion of the therapeutic film through the vaginal canal.

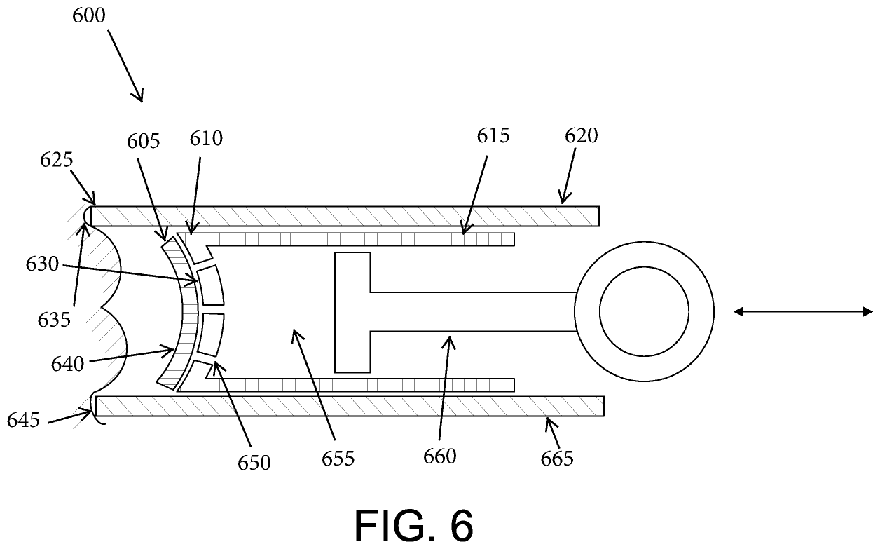

FIG. 6 depicts an insertion tool 600. The tool 600 includes a chamber 620 housing a wall 615 connected to a concave cup 610 of a radius approximately similar to the cervix radius. The cup includes spaces or is pierced and include airways 650 extending from the distal surface 630 or patch side of the cup and to a suction chamber 655. A plunger 660 causes a partial vacuum to form in the suction chamber when a therapeutic patch 605 is placed on the concave surface, and the plunger 660 is manipulated to pull a vacuum in the suction chamber via the airways 650.

The partial vacuum in the suction chamber will thus affix the therapeutic patch 605 to the concave surface 630, and permit the housing 620 to be inserted into the vaginal canal without exposing the therapeutic patch to the vaginal epithelium. The insertion tool 600 may be positioned such that distal end 625 of housing 620 is placed against the circular crease, upper portion 635 and a lower portion 645 of a patient, defined by intersection of the cervical epithelium and the vaginal epithelium, also known as the cervicovaginal junction. This permits precise positioning of the therapeutic patch against the cervical epithelium, and avoiding contact with the vaginal epithelium. The plunger 660 when depressed, will release the vacuum, thus releasing the therapeutic patch onto the cervix. The insertion tool is withdrawn leaving the therapeutic film in place. In some embodiments, the chamber 620 of the insertion tool tube may be replaced by using a speculum and guiding just the suction chamber walls 615 through the speculum. Embodiments may come in a variety of dimensions to enable matching to the variety of human sizes.

In some embodiments, the insertion tool may include a hollow projection shape, similar to the projection tips in FIG. 4, where the distal surface 630 of the cup (FIG. 6) is configured to place the therapeutic patch at least part way into the cervical opening, or the external os (the opening of the uterine cervix into the vagina). The therapeutic patch may be pre-attached and provided as patch and cup assembly. As an embodiment, the pre-attached therapeutic patch will have a protective peelable layer (not shown) covering the cervix side of the treatment layer. In an alternate embodiment, a therapeutic patch having the TCA dispersed in a radial concentration gradient or of radial thickness gradient (to create the radial concentration gradient) is pre-attached to the insertion tool to further concentrate treatment in the external os region.

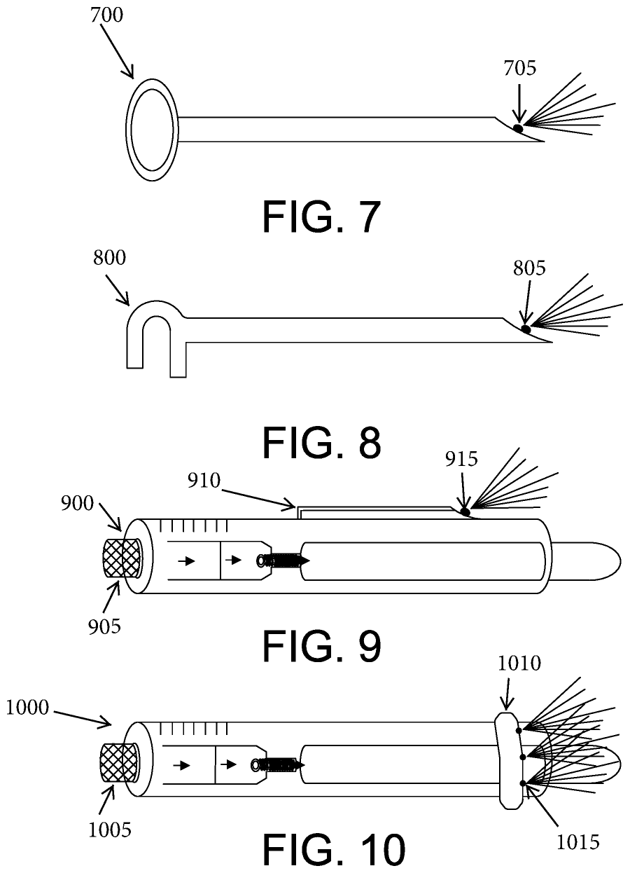

FIGS. 7-10 illustrate examples of different light emitting diode (LED) light attachments that may be clipped on to a pen shaped applicator, such as the applicators depicted in FIGS. 1-3, 5, and 6.

FIG. 7 illustrates a ring clip-on LED light attachment 700 with a light source that may allow the pen shaped applicator to pass through the ring. The LED light source at the end of the light attachment shines rays of light 705 to allow visualization and differentiation of the abnormally stained tissue from the normal tissue at the time of treatment.

FIG. 8 illustrates a C-shaped clip-on LED light attachment 800 with a light source that may attach on the side of the pen shaped applicator and uses the LED light source at the end of the light attachment shines rays of light 805 to allow visualization and differentiation of the abnormally stained tissue from the normal tissue at the time of treatment.

FIG. 9 illustrates a side attached light with a LED light source that may be attached to the side of clicker applicator 900 using an attachment 910, where the LED light source 915 on the attachment 910 shines rays of light from the side of the clicker applicator 900 to allow visualization and differentiation of the abnormally stained tissue from the normal tissue at the time of treatment. The LED 915 can be activated when the plunger or button 905 is pressed.

FIG. 10 illustrates an end-ring LED light 1010 that may be incorporated into the clicker applicator 1000 with a ring-like attachment 1010, containing multiple LED light sources, placed close to the tip of the clicker application. The LED light sources 1015 on the attachment 1010 shines rays of light from the end of the clicker applicator 1000 on to the abnormally stained tissue at the time of treatment for visualization and differentiation from the normal tissue. The LEDs 1010 can be activated when the plunger or button 1005 is pressed.

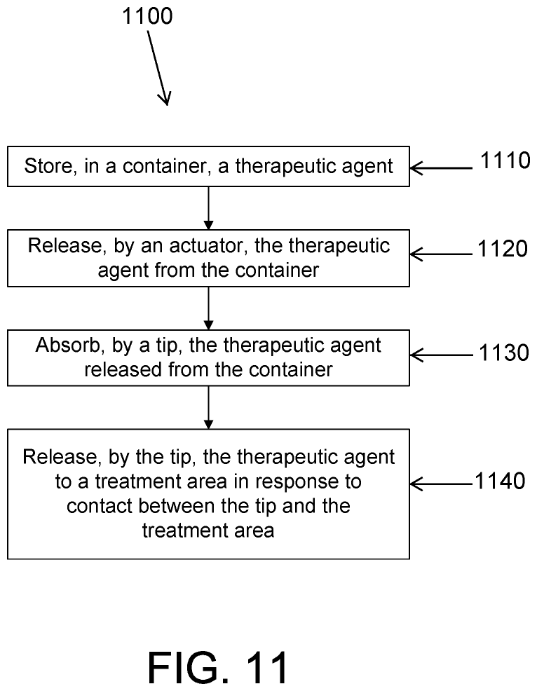

One method of performing an embodiment is found in FIG. 11, wherein a process 1100 for applying a therapeutic agent to a treatment area is illustrated. The first step in the process 1110 starts by storing a therapeutic agent in a container. The next step in the process 1120 is the release of the therapeutic agent from the container using an actuator. The following step in the process 1130 moves to absorption of the therapeutic agent by the tip which was released from the container. The final step in the process 1140 is releasing the therapeutic agent from the tip into the treatment area upon contact between the tip and the treatment area.

### Terminology

When a feature or element is herein referred to as being “on” another feature or element, it may be directly on the other feature or element or intervening features and/or elements may also be present. In contrast, when a feature or element is referred to as being “directly on” another feature or element, there may be no intervening features or elements present. It will also be understood that, when a feature or element is referred to as being “connected”, “attached” or “coupled” to another feature or element, it may be directly connected, attached or coupled to the other feature or element or intervening features or elements may be present. In contrast, when a feature or element is referred to as being “directly connected”, “directly attached” or “directly coupled” to another feature or element, there may be no intervening features or elements present.

Although described or shown with respect to one embodiment, the features and elements so described or shown may apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments and implementations only and is not intended to be limiting. For example, as used herein, the singular forms “a”, “an” and “the” may be intended to include the plural forms as well, unless the context clearly indicates otherwise. It will be further understood that the terms “comprises” and/or “comprising,” when used in this specification, specify the presence of stated features, steps, operations, processes, functions, elements, and/or components, but do not preclude the presence or addition of one or more other features, steps, operations, processes, functions, elements, components, and/or groups thereof. As used herein, the term “and/or” includes any and all combinations of one or more of the associated listed items and may be abbreviated as “/”.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

Spatially relative terms, such as “forward”, “backward”, “rearward”, “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the applicator in use or operation in addition to the orientation depicted in the figures. For example, if a device in the figures is inverted, elements described as “under” or “beneath” other elements or features would then be oriented “over” the other elements or features due to the inverted state. Thus, the term “under” may encompass both an orientation of over and under, depending on the point of reference or orientation. The device may be otherwise oriented (rotated 90 degrees or at other orientations) and the spatially relative descriptors used herein interpreted accordingly. Similarly, the terms “upwardly”, “downwardly”, “vertical”, “horizontal” and the like may be used herein for the purpose of explanation only unless specifically indicated otherwise.

Although the terms “first” and “second” may be used herein to describe various features/elements (including steps or processes), these features/elements should not be limited by these terms as an indication of the order of the features/elements or whether one is primary or more important than the other, unless the context indicates otherwise. These terms may be used to distinguish one feature/element from another feature/element. Thus, a first feature/element discussed could be termed a second feature/element, and similarly, a second feature/element discussed below could be termed a first feature/element without departing from the teachings provided herein.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise.

For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed.

It is also understood that throughout the application, data is provided in a number of different formats, and that this data, may represent endpoints or starting points, and ranges for any combination of the data points. For example, if a particular data point “10” and a particular data point “15” may be disclosed, it is understood that greater than, greater than or equal to, less than, less than or equal to, and equal to 10 and 15 may be considered disclosed as well as between 10 and 15. It is also understood that each unit between two particular units may be also disclosed. For example, if 10 and 15 may be disclosed, then 11, 12, 13, and 14 may be also disclosed.

In accordance with certain embodiments, the device is particularly implemented to be suitable for use in low-to-middle-income countries, where access to medical treatment is difficult, due to the complexity of the treatment options and the respective costs. Through this device, we aim to popularize a low-cost and effective method for the treatment of pre-cancer in low-to-middle-income countries where over 85% of the cervical cancer diagnoses occur.

In this application, numerous specific details are given to provide a thorough understanding of the invention. However, it will be apparent that the invention may be practiced without these specific details. In order to avoid obscuring the present invention, some well-known compositions, system configurations, and process steps are not disclosed in detail. The drawings showing embodiments of the system are semi-diagrammatic and not to scale and, particularly, some of the dimensions are for the clarity of presentation and are shown exaggerated in the drawing figures.

Where multiple embodiments are disclosed and described having some features in common, for clarity and ease of illustration, description, and comprehension thereof, similar and like features one to another will ordinarily be described with similar reference numerals. Although various illustrative embodiments have been disclosed, any of a number of changes may be made to various embodiments without departing from the disclosures herein.

For example, the order in which various described method steps are performed may be changed or reconfigured in different or alternative embodiments, and in other embodiments, one or more method steps may be skipped altogether. Optional or desirable features of various device and system embodiments may be included in some embodiments and not in others. Therefore, the foregoing description is provided primarily for the purpose of example and should not be interpreted to limit the scope of the claims and specific embodiments or particular details or features disclosed.

The examples and illustrations included herein show, by way of illustration and not of limitation, specific embodiments in which the disclosed subject matter may be practiced. As mentioned, other embodiments may be utilized and derived therefrom, such that structural and logical substitutions and changes may be made without departing from the scope of this disclosure. Such embodiments of the disclosed subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve an intended, practical or disclosed purpose, whether explicitly stated or implied, may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

The disclosed subject matter has been provided here with reference to one or more features or embodiments. Those skilled in the art will recognize and appreciate that, despite of the detailed nature of the example embodiments provided here, changes and modifications may be applied to said embodiments without limiting or departing from the generally intended scope. These and various other adaptations and combinations of the embodiments provided here are within the scope of the disclosed subject matter as defined by the disclosed elements and features and their full set of equivalents.

A portion of the disclosure of this patent document may contain material, which is subject to copyright protection. The owner has no objection to facsimile reproduction by any one of the patent documents or the patent disclosure, as it appears in the Patent and Trademark Office patent files or records, but reserves all copyrights whatsoever. Certain marks referenced herein may be common law or registered trademarks of the applicant, the assignee or third parties affiliated or unaffiliated with the applicant or the assignee. Use of these marks is for providing an enabling disclosure by way of example and shall not be construed to exclusively limit the scope of the disclosed subject matter to material associated with such marks.

1. An applicator for topically applying a therapeutic agent, the applicator comprising:
a container configured to store the therapeutic agent;
an actuator configured to release the therapeutic agent from the container; and
a tip configured to absorb the therapeutic agent released from the container, the tip further configured to release the therapeutic agent to a treatment area in response to contact between the tip and the treatment area.

2. The applicator of claim 1, wherein the therapeutic agent comprises at least one of a chemexfoliant, a soothing agent, or an organic agent.

3. (canceled)

4. (canceled)

5. (canceled)

6. (canceled)

7. (canceled)

8. The applicator of claim 1, further comprising a light source, the light source integrated within or attached to a body of the applicator.

9. (canceled)

10. The applicator of claim 1, further comprising a chemiluminescent light source.

11. The applicator of claim 81, further comprising a light source, the light source comprising a plurality of selectable spectral ranges, wherein the spectral ranges are configured to illuminate stained tissue for selective identification of tissue.

12. (canceled)

13. The applicator of claim 1, wherein the therapeutic agent comprises at least one of a liquid, a gel, or a patch.

14. The applicator of claim 1, wherein the tip comprises a porous, absorbent, and/or an adsorbent material.

15. The applicator of claim 1, wherein the tip comprises pores.

16. The applicator of claim 1, wherein the tip comprises at least one of a hygroscopic, hydrophilic, absorbent, or an adsorbent material, the tip configured to retain the therapeutic agent when the tip is not in contact with the treatment area.

17. The applicator of claim 1, wherein the tip comprises at least one of a dome-shaped, conical, cylindrical, chiseled, multiply curved or polygonal shape.

18. The applicator of claim 1, wherein the tip comprises a projection, the projection comprising a nipple or an extension, wherein a shape of the projection corresponds to a contour of the treatment area.

19. (canceled)

20. (canceled)

21. The applicator of claim 1, wherein the container comprises a capsule, the capsule comprising a frangible or puncturable material, the capsule configured to fracture or puncture in response to at least one of an applied circumferential pressure or an applied radial pressure.

22. (canceled)

23. The applicator of claim 1, further comprising an auger configured to control an amount of a linear pressure.

24. (canceled)

25. The applicator of claim 21, further comprising a puncture cap configured to puncture an end of the capsule.

26. The applicator of claim 1, wherein the container further comprises a tube configured to transport the therapeutic agent to the tip.

27. The applicator of claim 1, wherein the actuator comprises at least one of a twist end, a push end, a clicker, or a plunge end actuation mechanism.

28. The applicator of claim 1, wherein the container and the tip are configured for bidirectional flow of therapeutic agent between the container and the tip.

29. The applicator of claim 28, further comprising at least one of an absorbent material or a chamber configured to receive excess therapeutic agent from the tip.

30. The applicator of claim 29, wherein the chamber comprises an outer annulus, the outer annulus at least partially surrounding at least a portion of the tube.

31. The applicator of claim 29, wherein the actuator draws by suction the excess therapeutic agent from the tip.

32. The applicator of claim 31, wherein the actuator simultaneously releases the therapeutic agent from the container towards the tip and draws by suction the excess therapeutic agent from the tip.

33. The applicator of claim 31, wherein the actuator comprises:
a first actuator portion configured to release therapeutic agent from the container towards the tip; and
a second actuator portion configured to draw by suction the excess therapeutic agent from the tip.

34. The applicator of claim 33, further comprising a coupling, configured to link the first actuator portion with the second actuator portion, the coupling, configured to simultaneously cause the first actuator portion to move towards the tip and the second actuator portion to move away from the tip.

35. The applicator of claim 1, wherein the tip comprises a roller configured to control a release of fluid material.

36. The applicator of claim 1, wherein the container comprises a plurality of capsules configured to store the therapeutic agent, the actuator configured to release the therapeutic agent from each of the plurality of capsules independently and separately, the actuator configured to release the therapeutic agent to the treatment area from each of the plurality of capsules independently and separately.

37. (canceled)

38. (canceled)

39. A method for topically applying a therapeutic agent to a treatment area, the method comprising:
storing, in a container, a therapeutic agent;
releasing, by an actuator, the therapeutic agent from the container;
absorbing, by a tip, the therapeutic agent released from the container; and
releasing, by the tip, the therapeutic agent to a treatment area in response to contact between the tip and the treatment area.

40. The method of claim 39, further comprising absorbing at least some therapeutic agent from the treatment area.

41. The method of claim 39, further comprising suctioning at least some therapeutic agent from the treatment area.

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. An applicator for topically applying a therapeutic agent, the applicator comprising:
a container configured to store a film containing the therapeutic agent, wherein the film comprises the therapeutic agent; and
an actuator configured to hold and release the film from the container to a treatment area in response to contact between the film and the treatment area, wherein the film adheres to the treatment area upon contact with treatment area moisture, wherein the actuator comprises at least one of a plunger, a twist mechanism, or a clicker configured to generate a partial vacuum to hold the film on the applicator, and wherein the at least one of the plunger, the twist mechanism, and the clicker is configured to release the partial vacuum to release the film from the applicator.

78. (canceled)

79. (canceled)

80. (canceled)

81. (canceled)

82. (canceled)
